(19) United States
(12) Patent Application Publication
(10) Pub. No.: US 2020/0188392 A1
(43) Pub. Date: Jun. 18, 2020

(54) TREATMENT OF EPILEPSY WITH PLINABULIN OR HALIMADE OR DIKETOPIPERAZINE DERIVATIVES

(71) Applicants: Katholieke Universiteit Leuven, Leuven (BE); Technical University of Denmark, Kgs. Lyngby (DK)

(72) Inventors: Daniëlle COPMANS, Holsbeek (BE); Alexander CRAWFORD, Oslo (NO); Peter DE WITTE, Kessel-Lo (BE); Camila ESGUERRA, Oslo (NO); Sara KILDGAARD, Lyngby (DK); Thomas OSTENFELD LARSEN, Holte (DK); Annelii NY, Mol (BE)

(21) Appl. No.: 16/642,505

(22) PCT Filed: Aug. 28, 2018
(86) PCT No.: PCT/EP2018/073147

§ 371 Date: Feb. 27, 2020

(30) Foreign Application Priority Data
Aug. 28, 2017 (GB) 1713760.5

(51) Int. Cl. A61K 31/496; A61P 25/08

(52) U.S. Cl. CPC A61K 31/496; A61P 25/08

(57) ABSTRACT

The present invention discloses halimide and plinabulin and structural analogues and their use in the treatment and prevention in epilepsy and other seizures. The present invention further discloses methods to screen halimide-like molecules as pharmaceutically active compounds.

### FIELD OF THE INVENTION

The present invention provides a pharmaceutical composition for treating epilepsy.

### BACKGROUND OF THE INVENTION

Epilepsy is among the most common severe neurological conditions, affecting more than 70 million people worldwide [Sander (2003) Curr Opin Neurol 16, 165-170; Ngugi (2010) Epilepsia 51, 883-890; Singh & Trevick (2016), Neurol Clin 34, 837-847. It is characterized by an enduring predisposition of the brain to generate epileptic seizures, with neurobiologic, cognitive, psychological, and social consequences [Fisher et al. (2005) Epilepsia 46, 470-472]. The treatment of epilepsy consists mostly of pharmacotherapy with antiseizure drugs (ASDs) to control seizures [Golyala & Kwan (2017) Seizure 44, 147-156.]. Despite considerable efforts, current ASDs fail to control the seizures of 30% of patients due to drug-resistance [Dalic & Cook (2016) Neuropsychiatr Dis Treat 12, 2605-2616.]. Uncontrolled epilepsy can result in a poorer quality of life because of physical, psychological, cognitive, social, and occupational problems [Golyala & Kwan (2017) cited above; Blond et al. (2016) Neurol Clin 34, 395-410, viii.]. Moreover, first-line ASDs are associated with important adverse effects that can significantly impact daily life and are a main cause of treatment failure [Dalic & Cook (2016) cited above Neuropsychiatr Dis Treat 12, 2605-2616; Moshe et al. (2015) Lancet 385, 884-898; Cramer et al. (2010) Expert Rev Neurother 10, 885-891]. Hence, there is a high need for the development of safer ASDs that are more effective against drug-resistant seizures.

WO2008103916 discloses combinations therapies for cancer and neurogical disorders, wherein panaxytriol and a variety of tubulin binding agents are disclosed.

Zebrafish animal models for screening compounds for anti-epileptic activity have been described [e.g. MacRae and Peterson (2015) Nat Rev Drug Discov 14, 721-731].

### SUMMARY OF THE INVENTION

The present invention discloses halimide which was isolated from the bioactive marine-derived fungus Aspergillus insuetus. Halimide was found to have antiseizure activity in the larval zebrafish PTZ seizure model. Plinabulin was identified as a structural analogue of halimide and investigated for antiseizure activity in the larval zebrafish PTZ seizure model, the larval zebrafish EKP seizure model, and the mouse 6-Hz psychomotor seizure model.

Based on the prominent antiseizure activity in zebrafish, the present invention relates to halimide and plinabulin as compounds in the use for the treatment of drug-resistant focal seizures, and in the treatment of epilepsy in general.

The present invention accordingly relates to the screening of other halimide structural analogues and modified versions thereof for compounds which are suitable for the prevention and treatment of seizures.

The invention is summarized in the following statements:

1. Compounds comprising a 2,5 diketopiperazine group such as halimide or plinabulin for use in the treatment or prevention of epilepsy. Further examples are compound disclosed in Hayashi (2013) Chem. Pharm. Bull. 61, 889-901 and in U.S. Pat. No. 6,069,146 can be validated in the screening models of the present invention.

2. Halimide (as a mixture of enantiomers) or Halimide (the S-enantiomer) for use in the treatment or prevention of epilepsy.

3. A method for identifying a pharmaceutical compound against epilepsy, the method comprising the steps of:

providing a compound comprising a 2,5 diketopiperizane moiety, which moiety is substituted at the 6 position with a substituent comprising a imidazole moiety and which is substituted at the 3 position with a substituent comprising a benzyl moiety.

and testing the compound for antiseizure activity.

4. The method according to statement 3, wherein the pharmaceutical compound is a compound as depicted in FIG. 1, with modified molecular structure or stereochemistry.

5. The method according to statement 4, wherein antiseizure activity is determined in a zebrafish model.

6. The method according to statement 5, wherein antiseizure activity is further determined in a mammalian model.

7. The method according to any of statements 3 to 6, further comprising the step of testing the compound for a side effect.

8. The method according to any one of statements 3 to 7, further comprising the step of formulating a compound with determined antiseizure activity into a pharmaceutical composition with an acceptable carrier, for use in the treatment of epilepsy.

### DETAILED DESCRIPTION OF THE INVENTION

### Brief Description of the Figures

FIG. 1. Chemical structure of halimide and plinabulin.

FIG. 2. Antiseizure hit SK0107.

(A) Aspergillus insuetus IBT 28443 cultivated on Czapek Yeast extract agar (CYA) and Yeast extract sucrose agar (YES) media for 9 days at 25° C. in the dark. Base peak chromatograms of the crude extract and bioactive fraction SK0107 in positive electrospray ionization mode. (B, C) Antiseizure activity of SK0107 in the zebrafish pentylenetetrazole (PTZ) seizure model after 2 hours of incubation. PTZ-induced seizure-like behavior is expressed as mean actinteg units per 5 minutes (±SEM) during the 30 minutes recording period (B) and over consecutive time intervals (C). Means are pooled from three independent experiments with each 12 replicate wells per condition. Statistical analysis: (B) one-way ANOVA with Dunnett's multiple comparison test, (C) two-way ANOVA with Bonferroni posttests (GraphPad Prism 5). Significance levels: * p≤05; ** p≤01; *** p≤001. Abbreviation: vehicle, VHC.

FIG. 3. Bioactivity-guided identification of the active compounds of antiseizure hit SK0107.

(A) Aspergillus insuetus IBT 28443 cultivated on Czapek Yeast extract agar (CYA) media for 9 days in the dark at 25° C. Base peak chromatogram (BPC) of the most bioactive fraction (SK1312) from first reversed phase fractionation in positive electrospray ionization mode. BPC chromatograms of the two most bioactive fractions (SK1414 and SK1415) from the second reversed phase fractionation in positive electrospray ionization mode. UV and HRMS spectra for halimide (I). (B-D) Antiseizure activity of SK1312 (n=23-24 replicate wells per condition) (B), SK1414 (n=10-11 replicate wells per condition) (C), and SK1415 (n=22 replicate wells per condition) (D) in the zebrafish pentylenetetrazole (PTZ) seizure model after 2 hours of incubation at their maximum tolerated concentration (MTC), MTC/2, and MTC/4. PTZ-induced seizure-like behavior is expressed as mean actinteg units per 5 minutes (±SEM) during the 30 minutes recording period. (B, D) Data are pooled from two independent experiments with each 11-12 replicate wells per condition. (B-D) Statistical analysis: one-way ANOVA with Dunnett's multiple comparison test for comparison of sample+PTZ groups with vehicle (VHC)+PTZ control group, Kruskal-Wallis test with Dunn's multiple comparison test for comparison of sample+VHC groups with VHC+VHC control group (GraphPad Prism 5). Significance levels: * p≤05; ** p≤01; *** p≤001.

FIG. 4. Behavioral antiseizure analysis of halimide and plinabulin in the zebrafish PTZ seizure model.

Antiseizure activity of halimide (A, B) and plinabulin (C, D), in the zebrafish pentylenetetrazole (PTZ) seizure model after 2 hours of incubation, respectively. PTZ-induced seizure-like behavior is expressed as mean actinteg units per 5 minutes (±SEM) during the 30 minutes recording period (A, C) and over consecutive time intervals (B, D). (A, B) Means are pooled from three independent experiments with each 10-11 replicate wells per vehicle (VHC)+PTZ and compound+PTZ condition, and 9-11 replicate wells per VHC+VHC and compound+VHC condition. (C, D) 21-22 replicate wells for VHC+PTZ and VHC+VHC conditions, and 10-11 replicate wells for compound+PTZ condition. Statistical analysis: (A, C) one-way ANOVA with Dunnett's multiple comparison test, (B, D) two-way ANOVA with Bonferroni posttests (GraphPad Prism 5). Significance levels: * p≤05; ** p≤01; *** p≤001.

FIG. 5. Electrophysiological antiseizure analysis of halimide in the zebrafish PTZ seizure model.

Noninvasive local field potential recordings from the optic tectum of larvae pre-exposed to vehicle (VHC) and pentylenetetrazole (PTZ), VHC only, halimide and PTZ, or halimide and VHC. Larvae were incubated with 200 μg/mL halimide for 2 hours, conform with the maximum tolerated concentration and incubation time used in the behavioral assay. Epileptiform discharges are quantified by the number (mean±SD) (B) and cumulative duration (mean±SD) (C) of events per 10 minute recording. Larvae are considered to possess epileptiform brain activity when three or more events occurred during a 10 minute recording (A). Number of replicates per condition: 19 larvae were used for VHC+PTZ controls, 16 larvae were used for VHC+VHC controls, and 12 larvae were used for halimide+PTZ and halimide+VHC conditions. Statistical analysis: (A) Fisher's exact test with Bonferroni posttest, (B, C) Kruskal-Wallis test with Dunn's multiple comparison test (GraphPad Prism 5). Significance levels: * p≤05; ** p≤01; *** p≤001.

FIG. 6: Behavioral antiseizure analysis of halimide (3:1 R- and S-enantiomer mixture) and its enantiomers in the zebrafish PTZ seizure model. Antiseizure activity of halimide (mixture of enantiomers (3:1 R:S)) (A, B), the R-enantiomer of halimide (C, D), and the S-enantiomer of halimide (E, F) in the zebrafish pentylenetetrazole (PTZ) seizure model after 2 hours of incubation, respectively. PTZ-induced seizure-like behavior is expressed as mean actinteg units per 5 minutes (±SEM) during the 30 minutes recording period (A, C, E) and over consecutive time intervals (B, D, F). Data are pooled from two independent experiments with a total of 20 replicate wells for vehicle (VHC)+VHC, VHC+PTZ, and compound+PTZ conditions, and 11-12 replicate wells for compound+VHC conditions. Statistical analysis: (A, C, E) one-way ANOVA with Dunnett's multiple comparison test for comparison of compound+PTZ conditions with vehicle (VHC)+PTZ controls and Kruskal-Wallis test with Dunn's multiple comparison test for comparison of compound+VHC conditions with VHC+VHC controls, (B, D, F) two-way ANOVA with Bonferroni posttests (GraphPad Prism 5). Significance levels: * p≤05; ** p≤01; *** p≤001.

FIG. 7: Behavioral antiseizure analysis of plinabulin in the zebrafish PTZ seizure model and in the zebrafish EKP seizure model. Antiseizure activity of plinabulin in the zebrafish pentylenetetrazole (PTZ) seizure model (A) and in the zebrafish ethyl ketopentenoate (EKP) seizure model (B) after 2 hours of incubation. Seizure-like behavior is expressed as mean actinteg units per 5 minutes (±SEM) during the 30 minutes recording period. Data are pooled from three or four independent experiments. Number of replicates per condition: 77 larvae were used for vehicle (VHC)+VHC, VHC+PTZ or EKP, 3.13 μg/mL plinabulin+VHC, and 3.13 μg/mL plinabulin+PTZ or EKP conditions, 55 larvae were used for 12.5 μg/mL plinabulin+PTZ, and 33 or 44 larvae were used for all other plinabulin+VHC and plinabulin+PTZ or EKP conditions. Statistical analysis: Kruskal-Wallis test with Dunn's multiple comparison test (GraphPad Prism 5). Significance levels: * p≤05; ** p≤01; *** p≤001.

FIG. 8: Electrophysiological antiseizure analysis of plinabulin in the zebrafish PTZ seizure model and in the zebrafish EKP seizure model. Noninvasive local field potential recordings from the optic tectum of larvae pre-exposed to vehicle (VHC) and pentylenetetrazole (PTZ, A-C) or ethyl ketopentenoate (EKP, D-F), VHC only (A-F), plinabulin and PTZ (A-C) or EKP (D-F), or plinabulin and VHC (A-F). Larvae were incubated with 12.5 μg/mL plinabulin for 2 hours, conform with the highest test concentration and the incubation time used in the behavioral assay. Epileptiform discharges are quantified by the number (mean±SD) (B, E) and cumulative duration (mean±SD) (C, F) of events per 10 minute recording. Larvae are considered to possess epileptiform brain activity when three or more events occurred during a 10 minute recording (A, D). Number of replicates per condition: 15 and 16 larvae were used for VHC+PTZ and VHC+EKP controls, respectively, 15 larvae each were used for both VHC+VHC control conditions, 27 and 15 larvae were used for plinabulin+PTZ and plinabulin+EKP conditions, respectively, and 15 and 18 larvae were used for both plinabulin+VHC conditions (A-C and D-F, respectively). Statistical analysis: (A, D) Fisher's exact test with Bonferroni posttest, (B, C, E, F) Kruskal-Wallis test with Dunn's multiple comparison test (GraphPad Prism 5). Significance levels: * p≤05; ** p≤01; *** p≤001.

FIG. 9: Antiseizure activity analysis of plinabulin in the mouse 6-Hz psychomotor seizure model. Drug-resistant psychomotor seizures were induced by electrical stimulation (6 Hz, 0.2 ms rectangular pulse width, 3 s duration, 44 mA) through the cornea, 30 minutes after i.p. injection of vehicle (VHC, n=11), positive control valproate (n=6), or plinabulin (n=8-9). Mean seizure durations (±SD) are depicted. Statistical analysis: one-way ANOVA with Dunnett's multiple comparison test (GraphPad Prism 5). Significance levels: * p≤05; ** p≤01; *** p≤001.

### ABBREVIATIONS USED IN THE APPLICATION

ASD, antiseizure drug; CV, column volume; CYA, Czapek Yeast extract agar; dpf, days post-fertilization; DAD, diode array detection; DMSO, dimethyl sulfoxide; EKP, ethyl ketopentenoate; EtOAc, ethyl acetate; FA, formic acid; FDAA, 1-fluoro-2-4-dinitrophenyl-5-L-alanine amide; FP7, Seventh Framework Programme; LFP, local field potential; HCl, hydrogen chloride; MeCN, acetonitrile; MeOH, methanol; min, minute; MTC, maximum tolerated concentration; NP, natural product; PEG200, polyethylene glycol M.W. 200; PMR, photomotor response; PTZ, pentylenetetrazole; t_{1/2}, half-life; UHPLC-DAD-QTOFMS, Ultra-high performance liquid chromatography-diode array detection-quadrupole time of flight mass spectrometry; VHC, vehicle; YES, Yeast extract sucrose agar

### Definitions

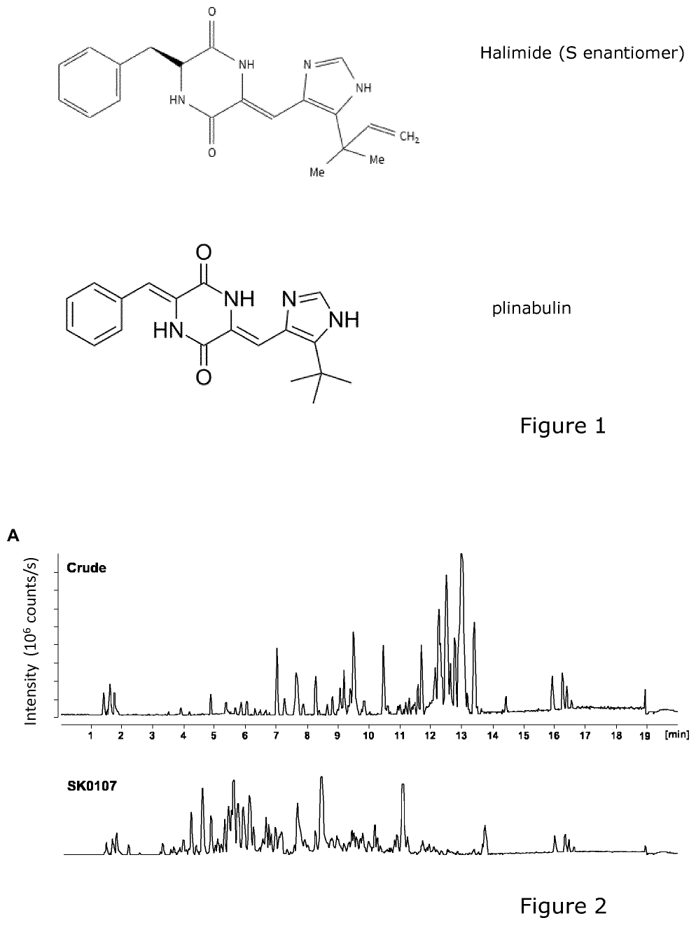

The present invention discloses compounds with a 2,5 diketopiperizane moiety. These are further substituted with substituents comprising a imidazole moiety and substituents comprising a benzyl moiety. Examples hereof are halimide (S enantiomer) and plinabulin, as depicted in FIG. 1.

Other compounds for use in the screening of candidate drugs against epilepsy are and which fall under the above definition are e.g. disclosed in Hayashi (2013) Chem. Pharm. Bull. 61, 889-901, U.S. Pat. No. 6,069,146, US200707138, US2004102545, WO2004054498, Kanoh et al (1999) Bioorg. Med. 7, 1451-1457

Explicit referral and incorporation by reference is made to compound screening of molecules with 2,5 diketopiperazine moiety as depicted in U.S. Pat. No. 6,069,146, and as recited in claim 1 of WO2004054498.

The above indicated medical use of the compounds equally comprises the use of the salt form thereof. Pharmaceutically acceptable salts include those described by Berge et al. J. Pharm. Sci. (1977) 66, 1-19.

Compounds are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. The corresponding stereospecific name and structure have been assigned to the final product where the enantiomeric excess of said product is greater than 70%. Assignment of absolute stereochemistry is based on the known chirality of the starting material. In examples where the composition of the final product has not been characterized by chiral HPLC, the stereochemistry of the final product has not been indicated. However, the chirality of the main component of the product mixture will be expected to reflect that of the starting material and the enatiomeric excess will depend on the synthetic method used and is likely to be similar to that measured for an analogous example (where such an example exists). Thus compounds shown in one chiral form are expected to be able to be prepared in the alternative chiral form using the appropriate starting material. Alternatively, if racemic starting materials are used, it would be expected that a racemic product would be produced and the single enatiomers could be separated by the usual methods. The invention also extends to any tautomeric forms and mixtures thereof.

“Seizure” refers to a brief episode of signs or symptoms due to abnormal excessive or synchronous neuronal activity in the brain. The outward effect can vary from uncontrolled jerking movement (tonic-clonic seizure) to as subtle as a momentary loss of awareness (absence seizure).

Seizure types are typically classified on observation (clinical and EEG) rather than the underlying pathophysiology or anatomy.

I Focal seizures (Older term: partial seizures)
IA Simple partial seizures—consciousness is not impaired
   IA1 With motor signs
   IA2 With sensory symptoms
   IA3 With autonomic symptoms or signs
   IA4 With psychic symptoms
IB Complex partial seizures—consciousness is impaired (Older terms: temporal lobe or psychomotor seizures)
   IB1 Simple partial onset, followed by impairment of consciousness
   IB2 With impairment of consciousness at onset
IC Partial seizures evolving to secondarily generalized seizures
   IC1 Simple partial seizures evolving to generalized seizures
   IC2 Complex partial seizures evolving to generalized seizures
   IC3 Simple partial seizures evolving to complex partial seizures evolving to generalized seizures

II Generalized seizures
IIA Absence seizures (Older term: petit mal)
   IIA1 Typical absence seizures
   IIA2 Atypical absence seizures
IIB Myoclonic seizures
IIC Clonic seizures
IID Tonic seizures,
IIE Tonic-clonic seizures (Older term: grand mal)
IIF Atonic seizures

III Unclassified epileptic seizures

A recent classification is described in Fisher et al. Operational classification of seizure types by the International League Against Epilepsy: Position Paper of the ILAE Commission for Classification and Terminology. Epilepsia (2017) 58, 522-30.

“Epilepsy” is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders or degenerative disease, head injury or trauma, metabolic disorders, cerebrovascular accident and alcohol withdrawal.

Treatment or prevention refers to any medical benefit from the patient, such as decreasing the frequency and severity of a seizure, or providing a therapy with less side effects or discomfort compared with existing therapies.

A large number of subtypes of epilepsy have been characterized and categorized. The classification and categorization system, that is widely accepted in the art, is that adopted by the International League Against Epilepsy's (“ILAE”) Commission on Classification and Terminology [See e.g., Berg et al. (2010) Epilepsia 51, 676-685]:

I. Electrochemical syndromes (arranged by age of onset):
I.A. Neonatal period: Benign familial neonatal epilepsy (BFNE), Early myoclonic encephalopathy (EME); Ohtahara syndrome
I.B. Infancy: Epilepsy of infancy with migrating focal seizures; West syndrome; Myoclonic epilepsy in infancy (MEI); Benign infantile epilepsy; Benign familial infantile epilepsy; Dravet syndrome; Myoclonic encephalopathy in non-progressive disorders
I.C. Childhood: Febrile seizures plus (FS+) (can start in infancy); Panayiotopoulos syndrome; Epilepsy with myoclonic atonic (previously astatic) seizures; Benign epilepsy with centrotemporal spikes (BECTS); Autosomal-dominant nocturnal frontal lobe epilepsy (ADNFLE); Late onset childhood occipital epilepsy (Gastaut type); Epilepsy with myoclonic absences; Lennox-Gastaut syndrome; Epileptic encephalopathy with continuous spike-and-wave during sleep (CSWS), also known as Electrical Status Epilepticus during Slow Sleep (ESES); Landau-Kleffner syndrome (LKS); Childhood absence epilepsy (CAE)
I.D. Adolescence-Adult: Juvenile absence epilepsy (JAE); Juvenile myoclonic epilepsy (JME); Epilepsy with generalized tonic-clonic seizures alone; Progressive myoclonus epilepsies (PME); Autosomal dominant epilepsy with auditory features (ADEAF); Other familial temporal lobe epilepsies
I.E. Less specific age relationship: Familial focal epilepsy with variable foci (childhood to adult); Reflex epilepsies

II. Distinctive constellations
II.A. Mesial temporal lobe epilepsy with hippocampal sclerosis (MTLE with
II.B. Rasmussen syndrome
II.C. Gelastic seizures with hypothalamic hamartoma
II.D. Hemiconvulsion-hemiplegia-epilepsy
II.E. Epilepsies that do not fit into any of these diagnostic categories, distinguished on the basis of presumed cause (presence or absence of a known structural or metabolic condition) or on the basis of primary mode of seizure onset (generalized vs. focal)

III. Epilepsies attributed to and organized by structural-metabolic causes
III.A. Malformations of cortical development (hemimegalencephaly, heterotopias, etc.)
III.B. Neurocutaneous syndromes (tuberous sclerosis complex, Sturge-Weber, etc.)
III.C. Tumor
III.D. Infection
III.E. Trauma

IV. Angioma
IV.A. Perinatal insults
IV.B. Stroke
IV.C. Other causes

V. Epilepsies of unknown cause

VI. Conditions with epileptic seizures not traditionally diagnosed as forms of epilepsy per se
VI.A. Benign neonatal seizures (BNS)
VI.B. Febrile seizures (FS)

A more recent classification is disclosed in Scheffer et al. ILAE classification of the epilepsies: Position paper of the ILAE Commission for Classification and Terminology. Epilepsia. (2017) 58, 512-521.

A first aspect of the present invention relates to halimide (S enantiomer) or plinabulin (depicted in FIG. 1) for use in the treatment or prevention of epilepsy, more particularly for preventing and alleviating seizures.

The examples of the present invention used specific compounds isolated from two Aspergillus strains. Herein some are effective in the zebrafish and mouse seizure models, while other show no pharmaceutical activity. The screening did not include chemically modified versions of halimide.

The present invention relates in another aspect to methods to identify other halimide structural analogues in the zebrafish and mouse model to identify compounds with a similar or higher activity than halimide or plinabulin, and or with better ADMET properties.

Candidate structural analogues of halimide described in the art are for example described in Hayashi (2013) Chem. Pharm. Bull. 61, 889-901 and in U.S. Pat. No. 6,069,146.

Methods of the present invention for drug screening can be advantageously performed with a zebrafish model which is suitable for large-scale screening and captures the complexity of a whole body organism and the central nervous system. As a vertebrate, zebrafish are highly similar to humans due to a high genetic, physiological and pharmacological conservation Moreover, given the small size of embryos and larvae, they fit in the well of microtiter plates and hence are suitable for medium to high-throughput testing. Given the low volumes used in 96- and 384-well plates, zebrafish larvae only require small amounts of sample in the low microgram range when added to their swimming water and even less when administered by injection. This property is of particular interest for marine natural product drug discovery, where material is often scarce. A particular suitable larval zebrafish seizure and epilepsy model, is the larval zebrafish pentylenetetrazole (PTZ) seizure model. This model has the following advantages 1) the model has been extensively characterized in terms of behavioral and non-behavioral seizure markers, 2) it has been pharmacologically characterized with ASDs on the market, 3) results translate well to rodent models, 4) seizures can easily and rapidly be induced by a single administration of the convulsant drug to the larva's aqueous environment, and 5) seizures can be quantified automatically by video recording [Baraban et al. (2005) Neurosci 131, 759-768; Afrikanova et al. (2013) PLoS One 8, e54166; Berghmans et al. (2007) Epilepsy Res 75, 18-28; Buenafe et al. (2013) ACS Chem Neurosci 4, 1479-1487; Orellana-Paucar et al. (2012) Epilepsy Behav 24, 14-22].

Within the framework of PharmaSea, halimide was isolated from the bioactive marine-derived fungus Aspergillus insuetus, which was isolated from a seawater trap set in the North Sea, in between Norway and Denmark. Halimide was investigated for antiseizure activity in the larval zebrafish PTZ seizure model and found to be active, after acute exposure. In addition, electrophysiological analysis from the zebrafish midbrain demonstrated that halimide also significantly lowered PTZ-induced epileptiform discharges. In addition, plinabulin was identified based on structural homology to halimide. Also plinabulin was demonstrated to have antiseizure activity in the larval zebrafish PTZ seizure model, after acute exposure. Moreover, plinabulin was found to be active in the zebrafish ethyl ketopentenoate (EKP) seizure model of drug-resistant seizures, suggesting activity against drug-resistant seizures [Zhang et al. (2017) Sci. Rep. 7, 7195. Finally, plinabulin showed antiseizure activity in a mammalian model of drug-resistant focal seizures, i.e. the mouse 6-Hz (44 mA) psychomotor seizure model.

Methods of the present invention for drug screening can further comprise the step of determining parameters such as absorption, distribution, metabolism, and excretion—toxicity.

In summary, based on the prominent antiseizure activity seen in zebrafish and mouse seizure models, the present invention claims halimide and plinabulin as compounds for use in the treatment of seizures.

### EXAMPLES

### Example 1. Methods

1.1. Chemical Experimental Procedures

Ultra-high performance liquid chromatography-diode array detection-quadrupole time of flight mass spectrometry (UHPLC-DAD-QTOFMS) was performed on an Agilent Infinity 1290 UHPLC system (Agilent Technologies, Santa Clara, Calif., USA) equipped with a diode array detector (DAD). Separation was achieved on an Agilent Poroshell 120 phenyl-hexyl column (2.1×150 mm, 2.7 μm) with a flow of 0.35 mL/min at 60° C. using a linear gradient 10% acetonitrile (MeCN) in Milli-Q water buffered with 20 mM formic acid (FA) increased to 100% in 15 min staying there for 2 min, returned to 10% in 0.1 min and kept there for 3 min before the following run. MeCN was LC-MS grade. MS detection was done on an Agilent 6550 iFunnel QTOF MS equipped with Agilent Dual Jet Stream electrospray ion source with the drying gas temperature of 160° C. and gas flow of 13 L/min and sheath gas temperature of 300° C. and flow of 16 L/min. Capillary voltage was set to 4000 V and nozzle voltage to 500 V. Data processing was performed using Agilent MassHunter Qualitative Analysis for quadrupole time of flight (version B.07.00). Pre-fractionation was performed using flash chromatography of the crude extract with an Isolera one automated flash system (Biotage, Uppsala, Sweden). Purification of compounds was conducted using a Waters 600 Controller (Milford, Mass., USA) coupled to a Waters 996 Photodiode Array Detector. One and two dimensional (1D and 2D) NMR experiments were acquired using standard pulse sequences on a 600 MHz Bruker Ascend spectrometer with a SmartProbe (BBO).

Optical rotations were measured on a Perkin Elmer 341 polarimeter (Perkin Elmer, Waltham, Mass., USA).

1.2. Microbial Strain and Microbial Cultivation

Aspergillus insuetus IBT 28443 was from the IBT culture collection at the Department of Biotechnology and Biomedicine, Technical University of Denmark. The fungus Aspergillus insuetus IBT 28443 was isolated from a seawater trap set in the North Sea, in between Denmark and Norway.

Aspergillus insuetus IBT 28443 was cultivated on one CYA and one YES media plates for 9 days in the dark at 25° C. for the original combined small scale extract. For the individual small scale extracts the fungus was cultivated on eight plates of CYA, eight plates of YES and eight plates of OAT for 9 days in the dark at 25° C.

For the large scale extract the fungus was cultivated on 250 plates of CYA for 9 days in the dark at 25° C.

Aspergillus ustus IBT 4133 was from the IBT culture collection at the Department of Biotechnology and Biomedicine, Technical University of Denmark.

Aspergillus ustus IBT 4133 was cultivated on 140 CYA media plate for 7 days in the dark at 25° C.

1.3. Microbial Extraction and Isolation

For the original combined small scale extract of Aspergillus insuetus IBT 28443 the two plates in total (one CYA and one YES) were extracted with 40 mL ethyl acetate (EtOAc) containing 1% FA. The crude extract was then fractionated on a reversed phase C₁₈ flash column (Sepra ZT, Isolute, 10 g) using an Isolera One automated flash system (Biotage, Uppsala, Sweden). The gradient used was 15%-100% MeCN buffered with 20 mM FA over 28 min (12 mL/min). Six flash fractions were automatically collected based on UV signal (210 nm and 254 nm). For the individual small scale extracts on CYA, YES and OAT each of the separate set of eight plates were extracted with 150 mL EtOAc with 1% FA and for the large scale extract on CYA it was extracted with 150 mL EtOAc with 1% FA for every 10 plates. All the crude extracts were fractionated on a reversed phase C₁₈ flash column (Sepra ZT, Isolute, 25 g/33 mL) using the Isolera One automated flash system. The gradient was 10% stepwise (12 column volumes) from 15% to 100% MeCN buffered with 20 mM FA with a flow of 25 mL/min. Fractions were collected manually for every 10%. For the large scale extract the most bioactive fraction (25% MeCN) was fractionated on a reversed phase Isolute SPE column (500 mg/3 mL) using methanol (MeOH) buffered with 20 mM FA. The compounds were eluted with 2 column volumes (CV) per fraction: 15% MeOH, 20% MeOH, 30% MeOH, 40% MeOH, 50% MeOH, 60% MeOH, 80% MeOH and 100% MeOH. From the 60% MeOH and 80% MeOH isolera fractions halimide separation was achieved on a Gemini C₆ Phenyl, 5 μm, 250×10 mm column (Phenomenex, Torrance, Calif., USA) with a flow of 4 mL/min. A linear gradient was used of 40% MeCN in Milli-Q water with 20 mM FA going to 70% MeCN in 30 min.

For the large scale cultivation of Aspergillus ustus IBT 4133, the 140 plates were extracted in seven 1 L beakers with 300 mL EtOAc per 20 plates. The EtOAc crude extract was fractionated on a reversed phase C₁₈ flash column (15 μm/100 Å, 25 g/33 mL) using the Isolera One automated flash system. MeCN and Milli-Q water was buffered with 20 mM FA and the flow was 25 mL/min. The gradient was stepwise from 15% to 100% MeCN and compounds were eluted with CV per fraction: 12 CV 15% MeCN, 6 CV 22% MeCN, 12 CV 25% MeCN, 6 CV 27% MeCN, 12 CV 30% MeCN, 12 CV 35% MeCN, 12 CV 65% MeCN and 12 CV 100% MeCN. Halimide purification was achieved from the 25% MeCN fraction on a Kinetex C₁₈, 5 μm, 250×10 mm column (Phenomenex, Torrance, Calif., USA) with a flow of 4 mL/min. A linear gradient was used of 25% MeCN in Milli-Q water with 20 mM FA going to 75% MeCN in 30 min.

Separation of the halimide enantiomers was achieved on a Lux Cellulose-1, 3 μm, 100×4.6 mm column (Phenomenex, Torrance, Calif., USA) with a flow of 2 mL/min and using a linear gradient of 20% MeCN in Milli-Q water going to 80% MeCN in 20 min.

Halimide (mixture): yellow solid; [α]_{D}⁺78 (c 0.24, MeOH); UV (MeCN) λmax: 205 nm; 236 sh nm; 320 nm. HRESIMS m/z 351.1818 [M+H]⁺ (calculated for C₂₀H₂₃N₄O₂, m/z 351.1816, Δ −0.77); R-enantiomer: [α]_{D}⁺ 213 (c 0.27, MeOH); S-enantiomer: [α]_{D}²⁰ 200 (c 0.09, MeOH)

TABLE 1
NMR spectroscopic data halimide
Halimide

| | δH (mult, J) | δC |
|---|---|---|
| 1 | — | — |
| 2 | 7.67 s | 135.1 |
| 3 | — | — |
| 4 | — | 132.4 |
| 5 | — | 138.7 |
| 6 | 6.60 s | 107.2 |
| 7 | — | 124.4 |
| 8 | — | — |
| 9 | — | 167.4 |
| 10 | 4.46 t(4.4) | 58.1 |
| 11 | — | — |
| 12 | — | 162.3 |
| 13 | 3.27 dd(13.6, 4.4) | 41.4 |
| | 3.06 dd(13.6, 4.4) | |
| 14 | — | 135.9 |
| 15/19 | 7.18 m | 131.5 |
| 16/18 | 7.20 m | 129.6 |
| 17 | 7.15 m | 128.4 |
| 20 | — | 38.8 |
| 21 | 5.99 dd(17.5, 10.6) | 146.6 |
| 22 | 5.02 dd(17.5, 1.0) | 113.1 |
| | 5.08 dd(10.6, 1.0) | |
| 23 | 1.41 d(6.6) | 28.6 |

NMR spectroscopic data (600 MHz, MeOD, δ in ppm, J in Hz) for halimide isolated from the crude extract of Aspergillus insuetus IBT 28443.

Marfey's Analysis

50 μg of halimide was hydrolyzed in 6 M hydrogen chloride (HCl) at 110° C. for 24 hours. After hydrolysis the sample was dried by a steam of N₂. To the hydrolysis product or L- and D-phenylalanine (2.5 μmol) was added 100 μL 0.125 M borate buffer and 100 μL 1% 1-fluoro-2-4-dinitrophenyl-5-L-alanine amide (FDAA) in acetone. This reaction was heated to 40° C. for 1 hour. The reaction was quenched by addition of 20 μL 1 M HCl and the solution was added 400 μL MeOH prior to UHPLC-DAD-QTOFMS analysis.

Commercial Standard

1.4. Compounds

Plinabulin was purchased at Adooq BioScience (Irvine, Calif. 92604, USA). Pentylenetetrazole (PTZ) and valproate were purchased from Sigma-Aldrich. EKP was synthesized as disclosed in Zhang et al. (cited above).

1.5. Compound and Sample Preparation

For experiments with zebrafish larvae, dry samples and compounds were dissolved in 100% dimethyl sulfoxide (DMSO, spectroscopy grade) as 100-fold concentrated stocks and diluted in embryo medium to a final concentration of 1% DMSO content, except for PTZ which was dissolved in embryo medium (0% DMSO). Control groups were treated with 1% DMSO (VHC) in accordance with the final solvent concentration of tested samples or compounds. For mice experiments, a mixture of poly-ethylene glycol M.W. 200 (PEG200) and 100% DMSO (spectroscopy grade) (1:1 PEG200:DMSO) was used as solvent and VHC.

1.6. Experimental Animals

All animal experiments carried out were approved by the Ethics Committee of the University of Leuven (approval numbers 101/2010, 061/2013, 150/2015, 023/2017, and 027/2017) and by the Belgian Federal Department of Public Health, Food Safety & Environment (approval number LA1210199).

Zebrafish

Adult zebrafish (Danio rerio) stocks of AB strain (Zebrafish International Resource Center, Oregon, USA) were maintained at 28.0° C., on a 14/10 hour light/dark cycle under standard aquaculture conditions. Fertilized eggs were collected via natural spawning and raised in embryo medium (1.5 mM HEPES, pH 7.2, 17.4 mM NaCl, 0.21 mM KCl, 0.12 mM MgSO4, 0.18 mM Ca(NO3)2, and 0.6 μM methylene blue) at 28.0° C., under constant light with regards to the zebrafish PTZ seizure model and under a 14/10 hour light/dark cycle with regards to the zebrafish photomotor response assay and the zebrafish EKP seizure model.

Mice

Male NMRI mice (weight 18-20 g) were acquired from Charles River Laboratories and housed in poly-acrylic cages under a 14/10-hour light/dark cycle at 21° C. The animals were fed a pellet diet and water ad libitum, and were allowed to acclimate for one week before experimental procedures were conducted. Prior to the experiment, mice were isolated in a poly-acrylic cage with a pellet diet and water ad libitum for habituation overnight in the experimental room, to minimize stress.

1.7. Zebrafish Photomotor Response Assay

Behavioral Analysis

Experiments were performed as described in Copmans et al. (2016) J Biomol Screen 21, 427-436.n the primary screen one replicate well was used per sample tested and each experimental plate contained 6 internal control wells. Each well held 5 embryos that were incubated with sample for 2 hours prior to behavioral recording at 32 hpf. A neuroactive hit was defined as a marine NP that modified the PMR such that its behavioral fingerprint (16 pseudo Z-scores that together describe the embryonic motion over a 30 second recording period) contained at least one pseudo Z-score with an absolute value equal to or exceeding 5.

Toxicity Evaluation

Each behavioral analysis was followed by visual evaluation of the embryos under a light microscope to assess toxicity of treatment. Overall morphology, heartbeat, and touch response were investigated. Marine NPs were scored normal or toxic. When embryos showed normal morphology, normal or lowered heartbeat, and normal or lowered touch response the treatment was considered to be normal. In case of an abnormal morphology and/or absence of touch response or heartbeat a treatment was considered to be toxic.

1.8. Zebrafish Pentylenetetrazole Seizure Model

Toxicity Evaluation

Maximum tolerated concentration (MTC) was determined prior to further experiments and used as the highest test concentration. Experiments were performed as described in Copmans et al. (2018) Neurochem. Int. 112, 124-133.before. In brief, the MTC was investigated by exposing 12 larvae of 6 or 7 dpf to a range of concentrations in a 100 μL volume during 18 hours. The following parameters were investigated after 2 and 18 hours of exposure: touch response, morphology, posture, edema, signs of necrosis, swim bladder, and heartbeat. MTC was defined as the highest concentration at which no larvae died nor showed signs of toxicity or locomotor impairment in comparison to VHC-treated control larvae. In case no MTC was reached, the highest soluble concentration was used.

For screening purposes, no MTC was determined, but behavioral analysis was followed by visual evaluation of the larvae under a light microscope to assess toxicity of treatment. Overall morphology, heartbeat, and touch response were investigated. Marine NPs were scored normal or toxic. When embryos showed normal morphology, heartbeat, and touch response the treatment was considered to be normal. In case of an abnormal morphology and/or absence of touch response or heartbeat a treatment was considered to be toxic.

Behavioral Analysis

Experiments were performed as described in Copmans et al. (2018) Neurochem. Int. 112, 124-133; Afrikanova et al (2013) PLoS One 8, e54166 and Orellana-Paucar et al. (2012) Epilepsy Behav 24, 14-22. In brief, a single 7 dpf larva was placed in each well of a 96-well plate and treated with either VHC (1% DMSO) or test compound (1% DMSO) in a 100 μL volume. Larvae were incubated in dark for 2 hours at 28° C., whereafter 100 μL of either VHC (embryo medium) or 40 mM PTZ (20 mM working concentration) was added to each well. Next, within 5 minutes the 96-well plate was placed in an automated tracking device (ZebraBox Viewpoint, France) and larval behavior was video recorded for 30 minutes. The complete procedure was performed in dark conditions using infrared light. Total locomotor activity was recorded by ZebraLab software (Viewpoint, France) and expressed in actinteg units, which is the sum of pixel changes detected during the defined time interval (5 minutes). Larval behavior was depicted as mean actinteg units per 5 minutes during the 30 minute recording period and over consecutive time intervals. Data are expressed as mean±SD for single experiments with regards to screening and as mean±SEM for single experiments and for independent experiments of which the means or data are pooled.

In the first secondary screen three replicate wells were used per sample (100 μg/mL) tested and each experimental plate contained 12 internal control wells. In the second secondary screen six replicate wells were used per sample and concentration tested (100, 33, and 11 μg/mL), again 12 internal control wells were used per experimental plate.

Electrophysiology

Non-invasive LFP recordings were measured from the midbrain (optic tectum) of 7 dpf zebrafish larvae pre-incubated with VHC only, PTZ only, compound and VHC, or compound and PTZ [Zdebik, et al. (2013) PLoS One 8, 6e10. Experiments were performed as described in Copmans, et al. (2018) Neurochem. Int. 112, 124-133 and Copmans et al. (2018) ACS chemical neuroscience.]. In brief, larvae were incubated for approximately 2 hours with VHC (1% DMSO) or test compound (1% DMSO) in a 100 μL volume. After incubation, an equal volume of VHC (embryo medium) or 40 mM PTZ (20 mM working concentration) was added to the well for 15 minutes prior to recording. These steps occurred at 28° C., while further manipulation and electrophysiological recordings occurred at room temperature (±21° C.). The larva was embedded in 2% low melting point agarose (Invitrogen) and the signal electrode (an electrode inside a soda-glass pipet (1412227, Hilgenberg) pulled with a DMZ Universal Puller (Zeitz, Germany), diameter ±20 microns, containing artificial cerebrospinal fluid (ACSF: 124 mM NaCl, 10 mM glucose, 2 mM KCl, 2 mM MgSO4, 2 mM CaCl₂), 1.25 mM KH2PO4, and 26 mM NaHCO₃, 300-310 mOsmols)) was positioned on the skin covering the optic tectum. A differential extracellular amplifier (DAGAN, USA) amplified the voltage difference between the signal (measured by the signal electrode) and the reference electrode. The differential signal was band pass filtered at 0.3-300 Hz and digitized at 2 kHz via a PCI-6251 interface (National instruments, UK) using WinEDR (John Dempster, University of Strathclyde, UK). A grounding electrode grounded the electrical system. All electrodes were connected with ACSF. Each recording lasted 600 seconds. Manual analysis was completed by quantification of the number, cumulative duration, and mean duration of epileptiform-like events with Clampfit 10.2 software (Molecular Devices Corporation, USA). An electrical discharge was considered epileptiform if it was a poly-spiking event comprising at least 3 spikes with a minimum amplitude of three times the baseline amplitude and a duration of at least 100 milliseconds. Data are expressed as mean±SD.

1.9. Zebrafish Ethyl Ketopentenoate Seizure Model

Toxicity Evaluation

Maximum tolerated concentration (MTC) was determined prior to further experiments and used as the highest test concentration. Experiments were performed as described in Copmans et al. (2018). Neurochem. Int. 112, 124-133. In brief, the MTC was investigated by exposing 12 larvae of 7 dpf to a range of concentrations in a 100 μL volume during 18 hours. The following parameters were investigated after 2 and 18 hours of exposure: touch response, morphology, posture, edema, signs of necrosis, swim bladder, and heartbeat. MTC was defined as the highest concentration at which no larvae died nor showed signs of toxicity or locomotor impairment in comparison to VHC-treated control larvae.

Behavioral Analysis

Experiments were performed as described in Zhang et al. (2017) Sci. Rep. 7, 7195. In brief, a single 7 dpf larva was placed in each well of a 96-well plate and treated with either VHC (1% DMSO) or test compound (1% DMSO) in a 100 μL volume. Larvae were incubated in dark for 2 hours at 28° C., whereafter 100 μL of either VHC (1% DMSO) or 1 mM EKP (1% DMSO, 500 μM working concentration) was added to each well. Next, within 5 minutes the 96-well plate was placed in an automated tracking device (ZebraBox Viewpoint, France) and larval behavior was video recorded for 30 minutes. The complete procedure was performed in dark conditions using infrared light. Total locomotor activity was recorded by ZebraLab software (Viewpoint, France) and expressed in actinteg units, which is the sum of pixel changes detected during the defined time interval (5 minutes). Larval behavior was depicted as mean actinteg units per 5 minutes during the 30 minute recording period and over consecutive time intervals. Data are pooled from independent experiments and expressed as mean±SEM.

Electrophysiology

Non-invasive LFP recordings were measured from the midbrain (optic tectum) of 7 dpf zebrafish larvae pre-incubated with VHC only, EKP only, compound and VHC, or compound and EKP. Larvae were incubated for approximately 2 hours with VHC (1% DMSO) or test compound (1% DMSO). After incubation, VHC (1% DMSO) or 1 mM EKP (1% DMSO, 500 μM working concentration) was added to the well for 15 minutes prior to recording. These steps occurred at 28° C., while further manipulation and electrophysiological recordings occurred at room temperature (±21° C.). The larva was embedded in 2% low melting point agarose (Invitrogen) and the signal electrode was positioned on the skin covering the optic tectum and electrophysiological recordings (room temperature) were performed as described above for the zebrafish PTZ seizure model and as described in Zhang et al. (2017) Sci. Rep. 7, 7195. Manual analysis was completed by quantification of the number, cumulative duration, and mean duration of epileptiform-like events with Clampfit 10.2 software (Molecular Devices Corporation, USA). An electrical discharge was considered epileptiform if it was a poly-spiking event comprising at least 3 spikes with a minimum amplitude of three times the baseline amplitude and a duration of at least 100 milliseconds. Data are expressed as mean±SD.

1.10. Mouse 6-Hz Psychomotor Seizure Model

Experiments were performed as previously described. In brief, NMRI mice (average weight 32 g, range 28-36 g) were randomly divided into control and treatment groups (n=6-11). 50 μL (injection volume was adjusted to the individual weight) of VHC (PEG200:DMSO 1:1) or treatment (an ASD or test compound dissolved in VHC) was i.p. injected in mice and after 30 minutes psychomotor seizures were induced by low frequency, long duration corneal electrical stimulation (6 Hz, 0.2 ms rectangular pulse width, 3 s duration, 44 mA) using an ECT Unit 5780 (Ugo Basile, Comerio, Italy). Mice were manually restrained and a drop of ocular anesthetic (0.5% lidocaine) was applied to the corneas before stimulation. Following electrical current stimulation, the mouse was released in a transparent cage for behavioral observation, which was video-recorded. VHC-treated mice typically displayed stun, twitching of the vibrissae, forelimb clonus, and Straub tail. In addition, facial and mouth jerking as well as head nodding were observed occasionally. Seizure durations were measured during the experiment by experienced researchers, familiar with the different seizure behaviors. In addition, seizure durations were determined by blinded video analysis to confirm or correct the initial observations. Data are expressed as mean±SD.

### Example 2. Zebrafish-Based Antiseizure Drug Discovery

2009 marine NPs, i.e., extracts and pre-fractionated fractions, provided by the different PharmaSea partners, were screened for neuroactivity at a concentration of 100 μg/mL (2 hours incubation time) using the zebrafish PMR assay. The PMR was described by a behavioral fingerprint of 16 pseudo Z-scores that represent the embryonic motion over a 30 second recording period using the first and third quantile (Q1 and Q3) for each of the 8 time periods. A neuroactive hit was defined as a marine NP that modified the PMR such that its behavioral fingerprint contained at least one pseudo Z-score with an absolute value equal to or exceeding 5. Each PMR-assay was followed by visual evaluation of the embryos under a light microscope to assess toxicity of treatment. Only 109 marine NPs were observed to cause toxicity. All other treatments did not induce toxicity under the test conditions, whereof 332 were neuroactive and 1568 samples were inactive. The 332 neuroactive hits underwent antiseizure analysis at a concentration of 100 μg/mL (2 hours incubation time) using the zebrafish PTZ seizure model. In this model the convulsant PTZ (20 mM) is administered to the swimming water of 7 days post-fertilization (dpf) larvae and induces typical seizure-like behavior that is characterized by high-speed swimming, whirlpool-like circling, clonus-like seizures, and loss of posture. An antiseizure hit was defined as a marine NP that significantly lowered the strongly elevated larval locomotion as a result of PTZ-induced seizures. Initially, 97 antiseizure hits were identified that did not result in toxicity, whereof 43 were confirmed in a second screen using twice the number of larvae per sample. Moreover, the latter screen investigated concentration-dependent effects by analyzing a three-fold serial dilution from 100 μg/mL onwards. Hit prioritization was based on efficacy, concentration-dependency, and sample availability.

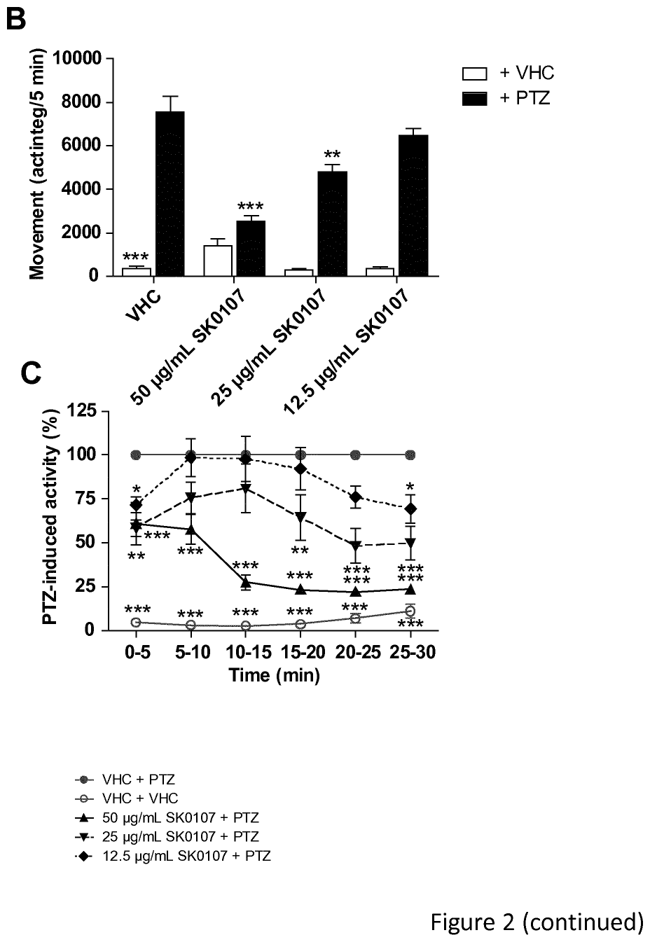

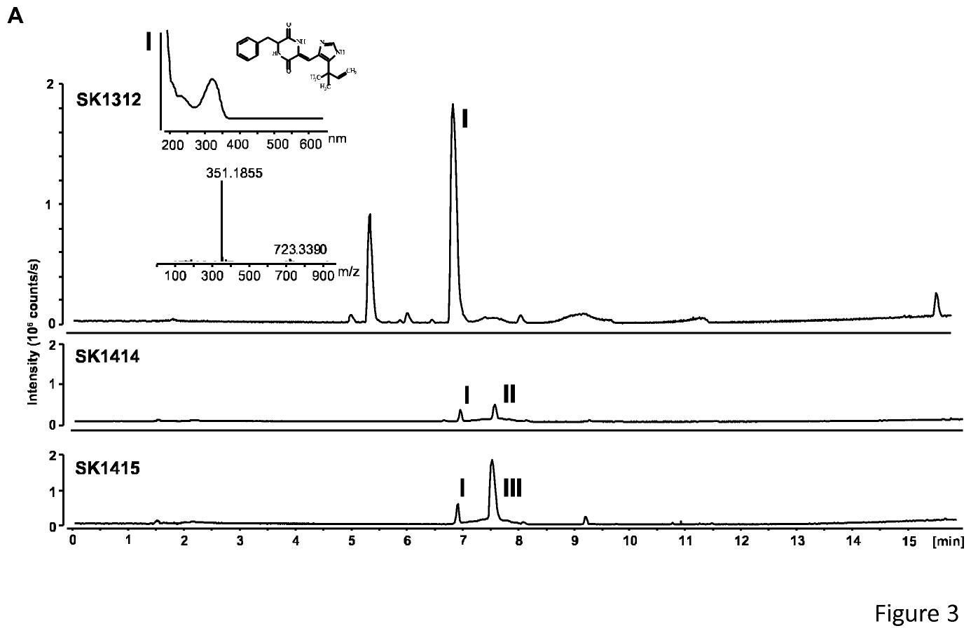

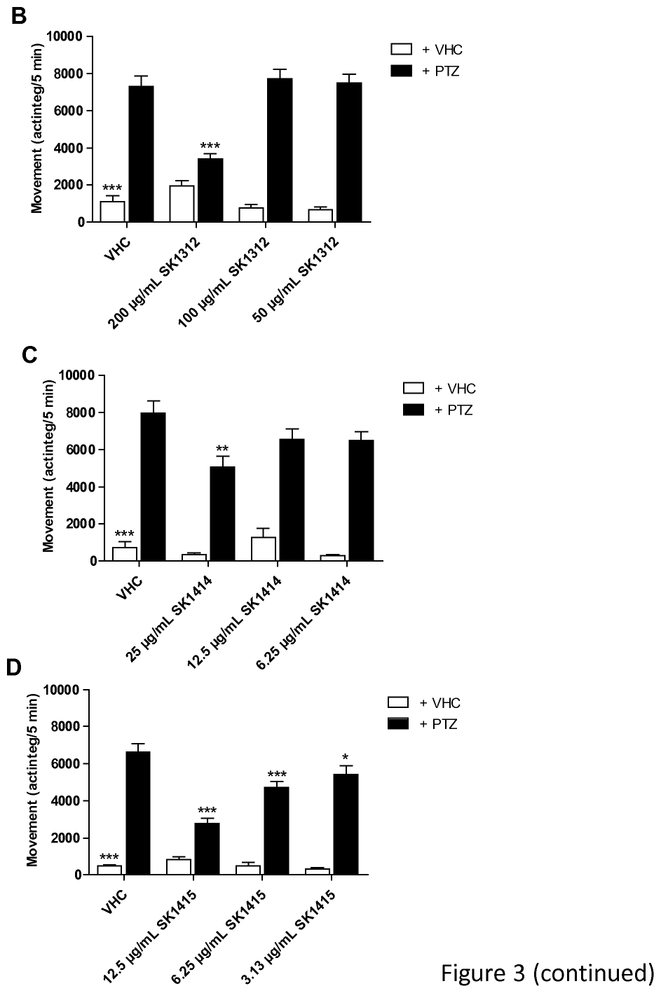

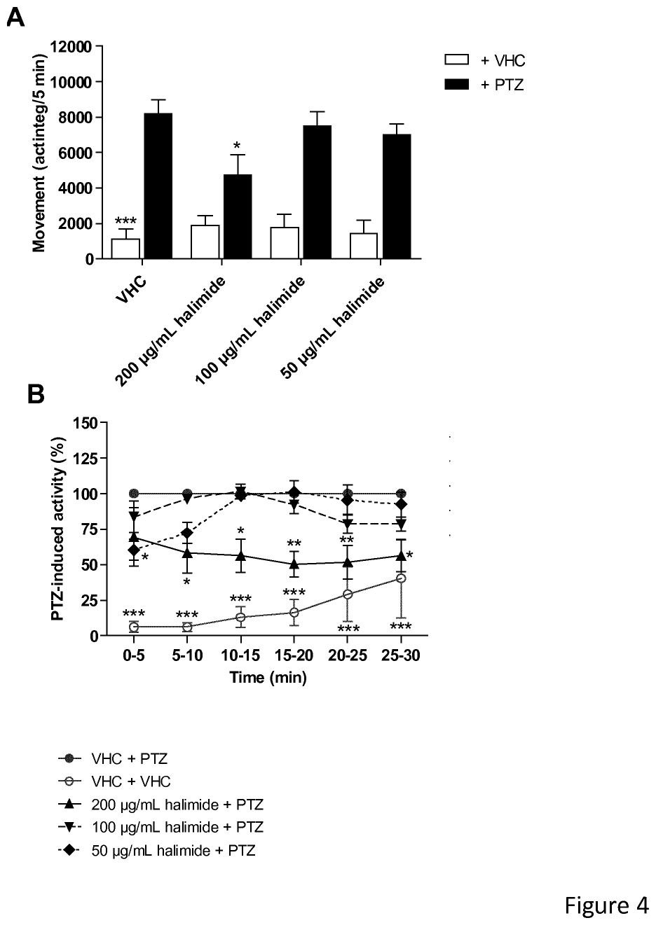

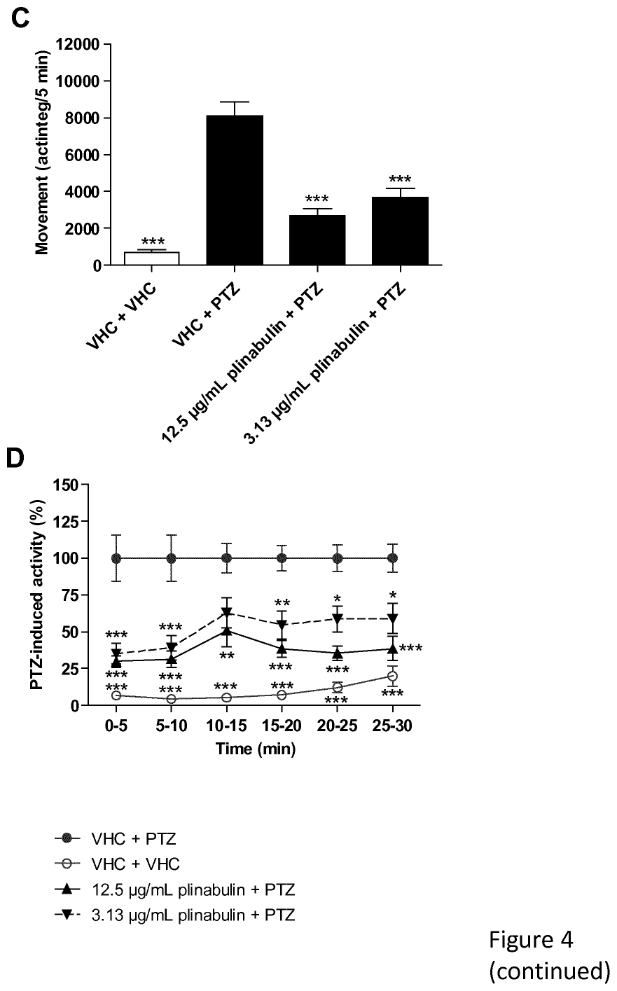

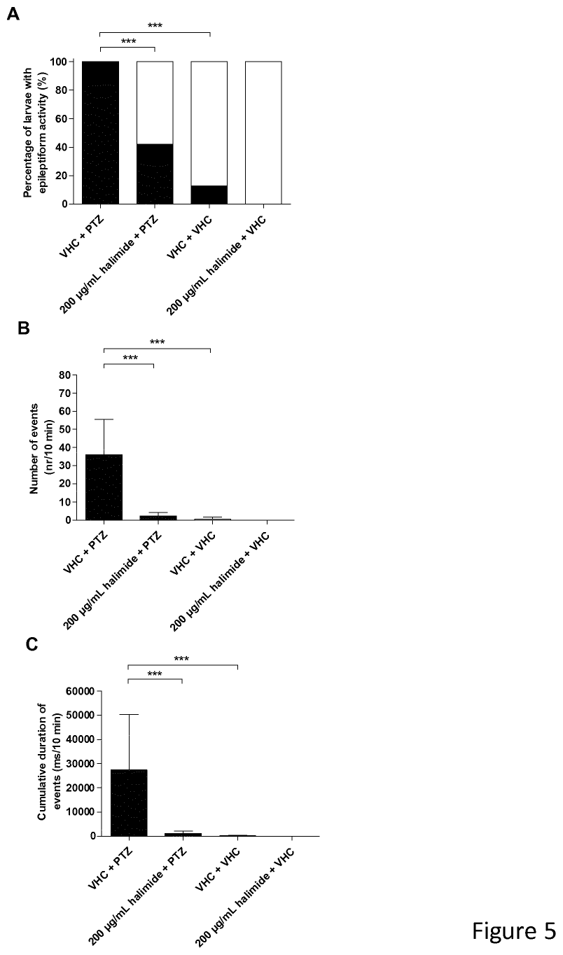

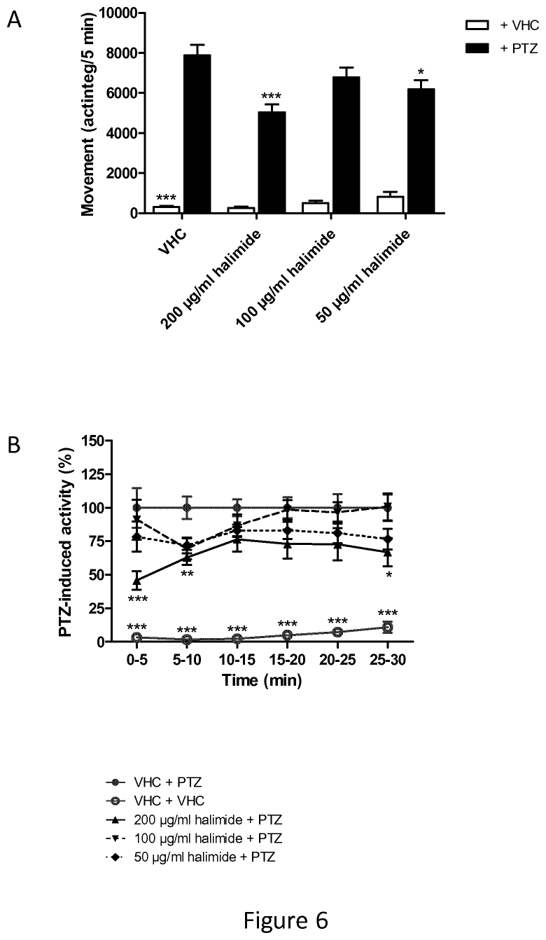

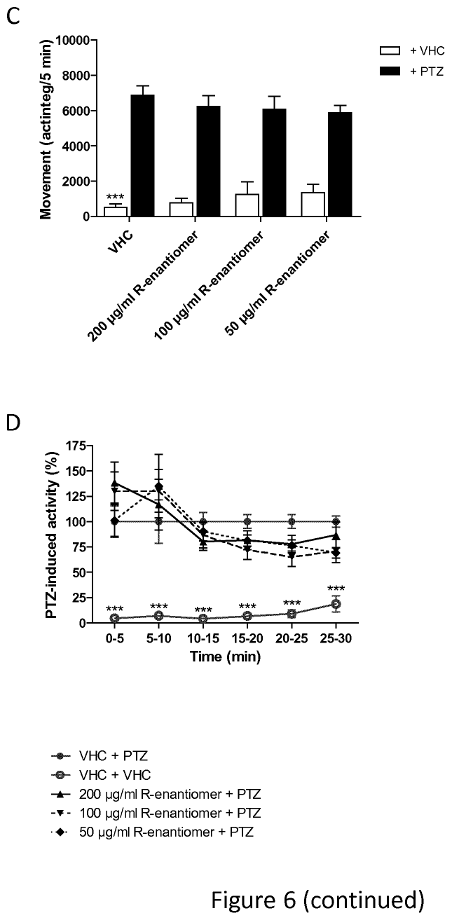

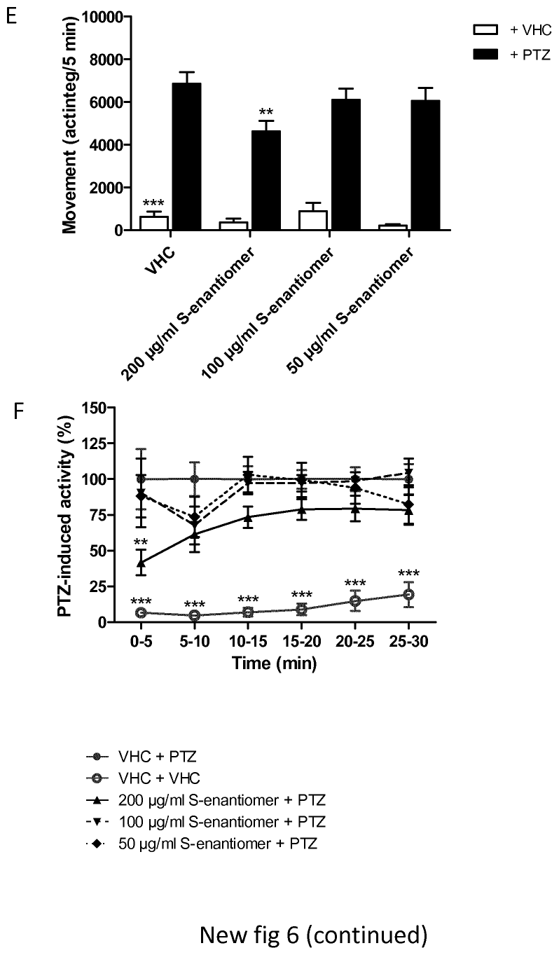

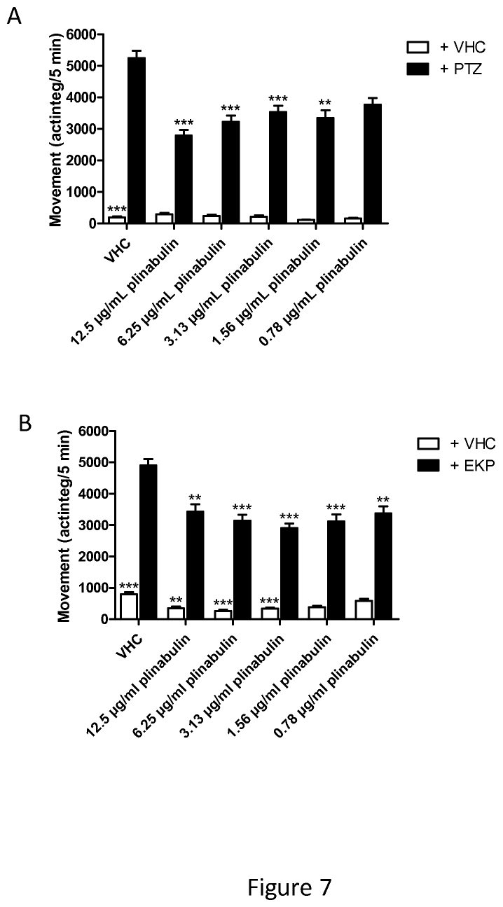

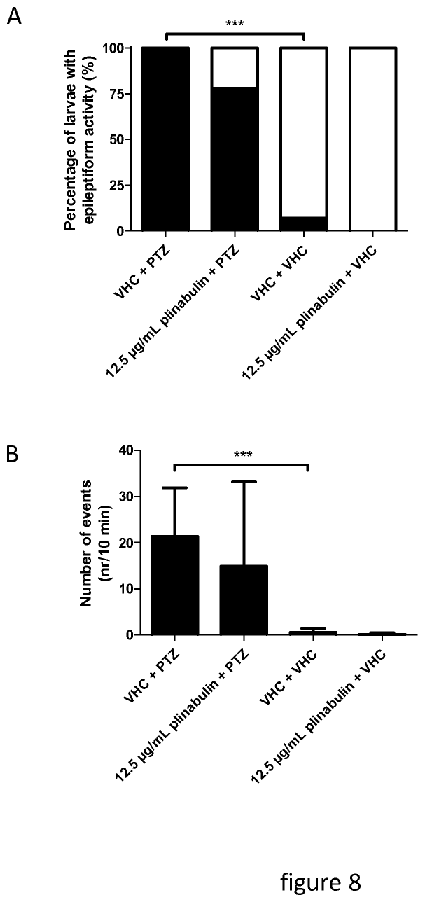

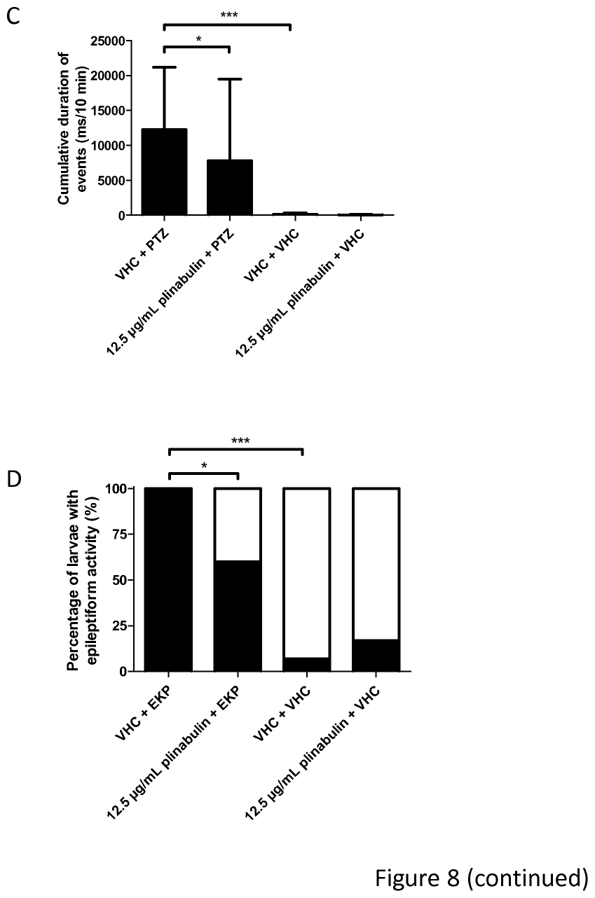

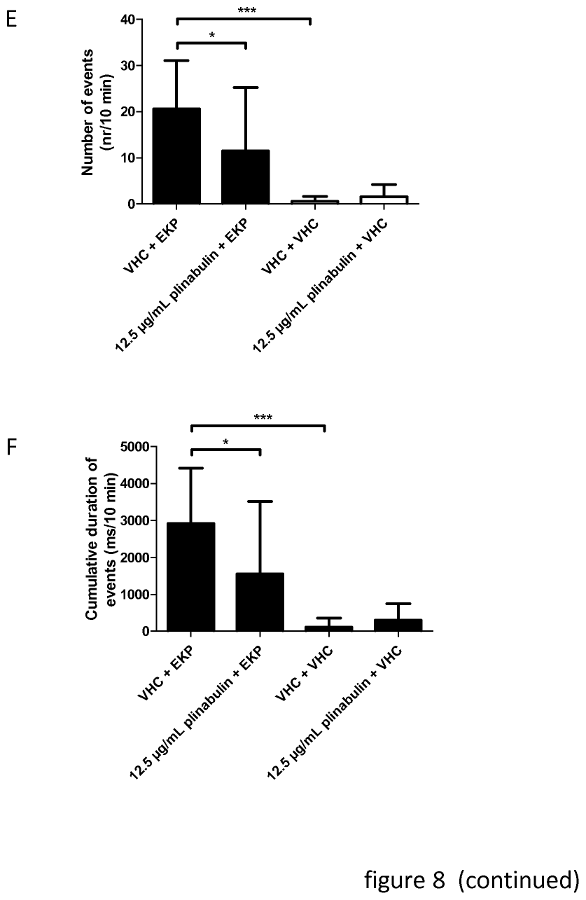

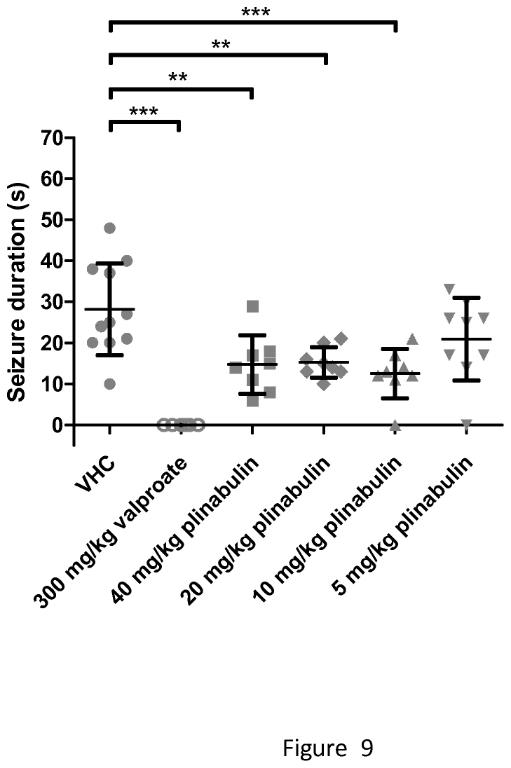

Among prioritized hits was marine NP SK0107, one of the more polar reversed phase fractions from the crude extract of Aspergillus insuetus IBT 28443 (FIG. 2A), which was isolated from a seawater trap set in the North Sea, in between Norway and Denmark. Aspergillus insuetus is a filamentous fungus belonging to Aspergillus section Usti that includes species from soil, foods, and indoor air environments but also from marine isolates. Marine-derived fungal isolates with Aspergillus species as a common source, have been seen to yield a plethora of biologically active compounds including structurally unique secondary metabolites. Prior to further experiments the maximum tolerated concentration (MTC) of SK0107 was determined, which was defined as the highest concentration at which no larvae died nor showed signs of toxicity or locomotor impairment in comparison to vehicle (VHC)-treated control larvae. The MTC was observed to be 50 μg/mL and used as the highest test concentration in all subsequent tests. To validate the results obtained during the course of screening the antiseizure activity of SK0107 was investigated in the larval zebrafish PTZ seizure model at the MTC, MTC/2, and MTC/4 (two-fold serial dilution, 2 hours incubation time) in three independent experiments (FIG. 2B-C). In line with former results, the antiseizure hit SK0107 showed significant concentration-dependent activity against PTZ-induced seizure behavior, both during the 30 minute (min) recording period (p≤001 and p≤01) (FIG. 2B) as over consecutive 5 min time intervals (p≤001, p≤01, and p≤05) (FIG. 2C).

### Example 3. Bioactivity-Guided Identification of Active Compounds

To identify the active constituents of SK0107 that are responsible for its antiseizure activity bioactivity-guided purification was performed of Aspergillus insuetus IBT 28443. In the crude extract of Aspergillus insuetus dereplication using ultra-high performance liquid chromatography-diode array detection-quadrupole time of flight mass spectrometry (UHPLC-DAD-QTOFMS) tentatively identified an abundant presence of the sesterterpenoids, ophiobolins (inactive, data not shown). Before any large scale cultivation, small scale extracts were prepared of the fungus cultivated individually on CYA, YES and OAT media, as the tested bioactive extract was of the combined cultivation on both CYA and YES media. This was done in hope of finding a medium where the production of ophiobolins was reduced and other compounds presented in a higher concentration than the original crude extract. CYA medium was chosen based on the activity of fractions from the crude extract and based on the reduced concentration of ophiobolins (data not shown).

A large scale extract was prepared from cultivation of Aspergillus insuetus IBT 28443 on CYA media for 9 days in the dark at 25° C. and bioactivity-guided purification was performed through several reversed phase purification steps until single compound isolation. In the two most bioactive fractions from the second fractionation of the crude extract, i.e., SK1414 and SK1415 (FIG. 3C-D), three compounds were tentatively identified by UHPLC-DAD-QTOFMS (FIG. 3A). One compound with the pseudomolecular ion, [M+H]⁺ m/z 351.1818 (mass accuracy −0.77 ppm) and two related compounds that were seen to coelute by first fractionation with the pseudomolecular ions, [M+H]⁺ m/z 242.1177 (mass accuracy −0.32 ppm) and m/z 240.1019 (mass accuracy 0.14 ppm). The molecular formula was based on the pseudomolecular ion for m/z 351.1818 established to be C₂₀H₂₂N₄O₂. A search in Antibase2012 for the formula revealed a possible candidate to be halimide (FIG. 3). This was supported by UV/Vis data consistent with litterature and production by related fungal species (Aspergillus ustus). The structure of halimide was confirmed by elucidation of the structure by 1D and 2D NMR spectroscopy and comparison of ¹H and ¹³C chemical shifts to literature data [Kanoh et al. (1997) Bioorg Med Chem Lett 7, 2847-2852].

In order to enable the further analysis and screening of halimide in the zebrafish PTZ seizure model various closely related species belonging to Aspergillus section Usti (Table 2) were investigated by HRMS, MS/HRMS and UV data analysis to find a better fungal producer. Aspergillus ustus IBT 4133 was chosen based on its production of halimide as the main compound and higher amounts were isolated (>15 mg).

TABLE 2
Potential halimide producing strains
from the Aspergillus section Usti.

| IBT number | Species |
|---|---|
| 4133 | Aspergillus ustus |
| 10619 | Aspergillus ustus |
| 28485 | Aspergillus insuetus |
| 914826 | Aspergillus calidoustus |

Closely related species belonging to Aspergillussection Usti from the IBT culture collection at the Department of Biotechnology and Biomedicine that are potential halimide producing strains.

Halimide was in this study discovered as a scalemic mixture based on the measurement of the optical rotation and Marfey's analysis, which suggested a ratio of about 3:1 amounts of the D and L phenylalanine residue. This is consistent with prior literature [Kanoh et al. (1997) Bioorg Med Chem Lett 7, 2847-2852].

In order to test the bioactivity of each enantiomer in the zebrafish PTZ seizure model chiral resolution was performed by chiral HPLC.

### Example 4. Halimide and Plinabulin Ameliorate Seizures in the Zebrafish PTZ Seizure Model

To confirm that halimide isolated from the most bioactive fractions was indeed the active constituent, its antiseizure activity was investigated in the zebrafish PTZ seizure model (FIG. 4A-B). Larvae were treated with halimide for 2 hours, using the MTC, MTC/2, and MTC/4, conform with the conditions used for the crude extract and purified fractions. Halimide significantly lowered PTZ-induced seizure behavior at its MTC in the 30 min recording period (p≤05, FIG. 4A). A more detailed analysis of the 30 min recording period into 5 min time intervals revealed a significant reduction of PTZ-induced seizure behavior during the entire time period (p≤01 and p≤05, FIG. 4B). These data demonstrate the antiseizure activity of halimide and confirm that the isolated compound is indeed an active constituent of the antiseizure hit SK0107, and the bioactive fractions SK1312, SK1414, and SK1415. The higher antiseizure efficacy of the bioactive extract and fractions in comparison to these observed for the individual compounds is possibly due to a synergistic action.

To investigate whether plinabulin, the commercially available structural analogue of halimide, has antiseizure activity, it was also tested in the zebrafish PTZ seizure model (FIG. 4C-D). Plinabulin significantly lowered PTZ-induced seizure behavior at the tested concentrations, i.e. 12.5 and 3.13 μg/mL, in the 30 min recording period (p≤0.001, FIG. 4C) and over consecutive time intervals (p≤0.001, p≤0.01, and p≤0.05, FIG. 4D). Hence, like halimide plinabulin has antiseizure activity.

### Example 5. Halimide Ameliorates Epileptiform Brain Activity in the Zebrafish PTZ Seizure Model

To determine whether halimide besides antiseizure activity also ameliorates the PTZ-induced hyperexcitable state of the brain that is characterized by epileptiform discharges, local field potential (LFP) recordings were non-invasively measured from the midbrain (optic tectum) of zebrafish larvae (FIG. 5). Larvae were treated with either VHC or test compound (MTC and 2 hours incubation time were used in line with previous experiments) followed by a 15 min during exposure to PTZ or VHC prior to LFP measurements. Pre-exposure to PTZ but not to VHC resulted in a significant increase of epileptiform electrical discharges. Pre-incubation with halimide significantly lowered the percentage of larvae with PTZ-induced epileptiform activity with almost 60% (p≤0.001) (FIG. 5A). A larva was considered to have epileptiform brain activity when at least 3 electrical discharges were seen during the 10 min recording that fulfilled the pre-defined requirements of an epileptiform event (see methods). In addition, pre-incubation with halimide significantly lowered the number (p≤001) and the cumulative duration (p≤001) of PTZ-induced epileptiform events over the 10 min recording period (FIGS. 5B and C). Thus, halimide shows anti-epileptiform activity and likely displays its antiseizure properties by counteracting the hyperexcitable state of the brain.

### Example 6. The S-Enantiomer of Halimide Ameliorates Seizures in the Zebrafish PTZ Seizure Model

The antiseizure activity of the separated enantiomers was tested alongside halimide (mixture of enantiomers (R:S=3:1)) in the zebrafish pentylenetetrazole (PTZ) seizure model (FIG. 6). This revealed the S-enantiomer to be active (p<0.01, FIG. 6E-F), whereas the R-enantiomer displayed no significant effect (FIG. 6C-D). These data suggest that the activity of the mixture of enantiomers, as observed in FIGS. 4A-B, 5, and 6A-B, is due to the S-enantiomer of halimide. However, there is likely a synergetic action between the R- and S-enantiomer with regards to the antiseizure activity of halimide because the activity of the S-enantiomer alone was only significant at a concentration that is fourfold higher, i.e. 200 μg/mL (FIG. 6E-F), than its actual concentration in the halimide mixture of enantiomers, i.e. 50 μg/mL S-enantiomer was approximately present within 200 μg/mL halimide (R:S=3:1) tested (FIG. 6A-B).

### Example 7. Plinabulin Ameliorates Seizures in the Zebrafish PTZ Seizure Model and in the Zebrafish EKP Seizure Model of Drug-Resistant Seizures

Plinabulin, the commercially available structural analogue of halimide, was tested in the zebrafish pentylenetetrazole (PTZ) seizure model [Baraban et al. Neuroscience (2005) 131, 759-68; Afrikanova et al. PLoS One (2013) 8, e54166.] as well as in the zebrafish ethyl ketopentenoate (EKP) seizure model of drug-resistant seizures [Zhang et al. Sci Rep (2017) 7, 7195] to determine whether plinabulin has antiseizure activity like halimide, and whether it would be effective against EKP-induced drug-resistant seizures (FIG. 7). Larvae were treated with 0.78-12.5 μg/mL plinabulin for 2 hours whereafter VHC, PTZ or EKP was administered prior to behavioral video recording. Plinabulin significantly lowered PTZ-induced seizure behavior at 1.56-12.5 μg/mL in the 30 min recording period (p≤0.01 (1.56 μg/mL) and p≤0.001 (3.13, 6.25, and 12.5 μg/mL)) (FIG. 7A), thereby demonstrating that plinabulin has antiseizure activity. Moreover, plinabulin significantly lowered EKP-induced seizure behavior at all concentrations tested in the 30 min recording period (p≤0.01 (0.78 and 12.5 μg/mL) and p 0.001 (1.56, 3.13, and 6.25 μg/mL)) (FIG. 7B), thereby demonstrating that plinabulin is active against EKP-induced drug-resistant seizures. Hence, plinabulin could have potential to treat drug-resistant seizures.

Of note, 3.13, 6.25, and 12.5 μg/mL plinabulin significantly lowered the normal swimming behavior in comparison to VHC-treated larvae in the zebrafish EKP seizure model (FIG. 7B), but not in the zebrafish PTZ seizure model (FIG. 7A).

### Example 8 Plinabulin Ameliorates Epileptiform Brain Activity in the Zebrafish PTZ Seizure Model and in the Zebrafish EKP Seizure Model of Drug-Resistant Seizures

To determine whether plinabulin besides antiseizure activity also ameliorates the PTZ- and/or EKP-induced hyperexcitable state of the brain that is characterized by epileptiform discharges, local field potential (LFP) recordings were non-invasively measured from the midbrain (optic tectum) of zebrafish larvae (FIG. 8) [Britton et al In Electroencephalography (EEG): An Introductory Text and Atlas of Normal and Abnormal Findings in Adults, Children, and Infants, St. Louis, E. K.; Frey, L. C., Eds. Chicago, (2016) Zdebik et al. PLoS One (2013) 8, e79765]. Larvae were treated with VHC or 12.5 μg/mL plinabulin for 2 hours followed by either a 15 min during exposure to PTZ or VHC with regards to the zebrafish PTZ seizure model, or by a 15 min during exposure to EKP or VHC with regards to the zebrafish EKP seizure model, prior to LFP measurements.

Pre-incubation with plinabulin only non-significantly lowered the percentage of larvae with PTZ-induced epileptiform activity (FIG. 8A). A larva was considered to have epileptiform brain activity when at least 3 electrical discharges were seen during the 10 min recording that fulfilled the pre-defined requirements of an epileptiform event (see methods). Plinabulin also non-significantly lowered the number of PTZ-induced epileptiform events within the 10 min recording period (FIG. 8B), but significantly lowered the cumulative duration (p≤0.05) of PTZ-induced epileptiform events over the 10 min recording period (FIG. 8C). Thus, plinabulin ameliorates the PTZ-induced hyperexcitable state of the brain. Pre-incubation with plinabulin significantly (p≤0.05) lowered the percentage of larvae with EKP-induced epileptiform activity by 40% (FIG. 8D). Again, a larva was considered to have epileptiform brain activity when at least 3 electrical discharges were seen during the 10 min recording that fulfilled the pre-defined requirements of an epileptiform event (see methods). Plinabulin also significantly lowered the number (p≤0.05, FIG. 8E), and the cumulative duration (p≤0.05, FIG. 8F) of EKP-induced epileptiform events over the 10 min recording period. Thus, plinabulin also ameliorates the EKP-induced hyperexcitable state of the brain. Taken together, these data demonstrate that besides antiseizure activity plinabulin also has anti-epileptiform activity in both the zebrafish PTZ seizure model as in the zebrafish EKP seizure model.

### Example 9 Plinabulin Ameliorates Focal Seizures in the Mouse 6-Hz (44 mA) Psychomotor Seizure Model

Despite the high genetic, physiological and pharmacological conservation, zebrafish are more distinct from humans than mammals [MacRae Peterson, R. T., Nat Rev Drug Discov (2015) 14, 721-731; Wilcox et al. Epilepsia (2013), 54 S4, 24-34]. Therefore, we wanted to investigate whether the antiseizure action of plinabulin observed in the larval zebrafish model translates to a standard rodent seizure model. From the available rodent seizure models we chose the mouse 6-Hz (44 mA) psychomotor seizure model, a gold standard in current ASD discovery efforts that is useful for screening and can detect compounds with novel antiseizure mechanisms and with potential against drug-resistant seizures. It is an acute model of drug-resistant focal impaired awareness seizures, previously referred to as complex partial or psychomotor seizures, that are induced by a low frequency, long duration corneal electrical stimulation. [Barton et al. Epilepsy Res (2001) 47, 217-27; Kehne et al. Neurochem Res (2017); Fisher et al. Epilepsia (2017) 58, 531-542; Holcomb & Dean Psychomotor Seizures. In Encyclopedia of Child Behavior and Development, Goldstein, S.; Naglieri, J. A., Eds. Springer US: Boston, Mass., (2011); pp 1191-1192]

Male NMRI mice were intraperitoneally (i.p.) injected with a 50 μL volume (adjusted to the individual weight) of VHC (DMSO:PEG200 1:1), positive control valproate (300 mg/kg), or plinabulin (40, 20, 10, and 5 mg/kg) 30 min before electrical stimulation (FIG. 9). VHC injected mice showed characteristic seizure behavior with a mean (±SD) duration of 28 seconds (s) (±11 s). In line with previous studies, mice that were injected with valproate were fully protected against the induced seizures [Orellana-Paucar et al. PLoS One (2013), 8, e81634] as none of the mice showed any seizure after electrical stimulation (p<0.001). Mice i.p. injected with plinabulin had a shorter seizure duration than the VHC control group, which was significant at 40 mg/kg (p<0.05, mean duration of 15 s (±7 s)), 20 mg/kg (p<0.01, mean duration of 15 s (±4 s)), and 10 mg/kg (p <0.05, mean duration of 12.5 s (±6 s)), but not at 5 mg/kg (mean duration of 21 s (±10 s)). Thus, the antiseizure activity of plinabulin that was observed in the larval zebrafish PTZ seizure model translates to a standard mouse model of drug-resistant focal seizures, thereby demonstrating the effectiveness of our zebrafish-based ASD discovery approach and the potential of marine NPs. Moreover, these observations confirm the translation of findings from zebrafish larvae to mice in the field of epilepsy, as previously published [Buenaf et al. ACS Chem Neurosci (2013) 4, 1479-87; Orellana-Paucar et al. Epilepsy Behav (2012) 24, 14-22].

1. Halimide or plinabulin for use in the treatment or prevention of epilepsy.

2. The halimide for use in the treatment or prevention of epilepsy in accordance with claim 1, wherein the halimide is the S enantiomer.

3. A method for identifying a pharmaceutical compound against epilepsy, the method comprising the steps of:
providing a compound comprising a 2,5 diketopiperazine moiety, which moiety is substituted at the 6 position with a substituent comprising a imidazole moiety and which is substituted at the 3 position with a substituent comprising a benzyl moiety,
and testing the compound for antiseizure activity.

4. The method according to claim 3, wherein antiseizure activity is determined in a zebrafish model.

5. The method according to claim 3, wherein antiseizure activity is further determined in a mammalian model.

6. The method according to claim 3, further comprising the step of testing the compound for a side effect.

7. The method according to claim 3, further comprising the step of formulating a compound with determined antiseizure activity into a pharmaceutical composition with an acceptable carrier, for use in the treatment of epilepsy.
